(19) United States
(12) Patent Application Publication
(10) Pub. No.: US 2011/0064760 A1
(43) Pub. Date: Mar. 17, 2011

(54) POLYPEPTIDES COMPRISING EPITOPES OF HIV GP41 AND METHODS OF USE

(76) Inventors: Michael W. Cho, Ames, IA (US); Dong P. Han, Ames, IA (US); Keiji Takamoto, West Orange, NJ (US)

(21) Appl. No.: 12/857,210

(22) Filed: Aug. 16, 2010

Related U.S. Application Data
(60) Provisional application No. 61/233,881, filed on Aug. 14, 2009.

(51) Int. Cl. A61K 39/21; C12P 21/00; A61P 31/18; A61P 37/04

(52) U.S. Cl. 424/188.1; 424/208.1; 435/69.3

(57) ABSTRACT

An isolated polypeptide that has an amino acid sequence that is substantially homologous to consecutive amino acids of a c-terminal portion of the membrane proximal external region (MPER) of gp41 includes an immunogenic epitope reactive with at least one anti-HIV antibody.

### RELATED APPLICATION

This application claims priority from U.S. Provisional Application No. 61/233,881, filed Aug. 14, 2009, the subject matter which is incorporated herein by reference.

### TECHNICAL FIELD

This application relates generally to polypeptides comprising epitopes of the HIV-1 gp41 subunit, and more particularly to soluble polypeptides containing the membrane proximal external region of gp41.

### BACKGROUND OF THE INVENTION

The Human Immunodeficiency Virus (HIV) is the causative agent of Acquired Immunodeficiency Syndrome (AIDS). HIV rapidly undergoes genetic changes to escape from the subject's immune system response. Identification of potent, broadly cross-reactive human monoclonal antibodies to HIV has major implications for development of HIV inhibitors, vaccines, and tools for understanding mechanisms of HIV entry.

The binding of the HIV-1 envelope glycoprotein to CD4 and co-receptors initiates a series of conformational changes that lead to viral entry into cells. HIV-1 envelope glycoprotein (Env) is composed of two subunits, gp120 and gp41. gp120 is highly variable and immunogenic. gp41 is conserved, but unstable in the absence of the other subunit, gp120. Screening of immune human antibody phage libraries by using purified soluble gp140s that contain both gp120 and truncated gp41 lacking the transmembrane domain and cytoplasmic tail often leads to the selection of antibodies against gp120.

A successful HIV vaccine must be able to induce broadly cross-reactive neutralizing antibodies against the large diversity of HIV-1 isolates that exist. Current research suggest that one target for developing an antigen that could elicit such humoral immunity is the membrane proximal external region (MPER) of HIV-1 gp41. The MPER is a highly conserved region targeted by two known monoclonal antibodies (i.e., 2F5 and 4E10) that exhibit broadly cross-reactive neutralizing activity. Unfortunately, generating a soluble protein or protein fragments containing gp41 MPER has been a major hurdle and remains a critical roadblock to developing an effective HIV vaccine.

### SUMMARY OF THE INVENTION

This application relates generally to isolated polypeptides that have an amino acid sequence that is substantially homologous to epitopes of the HIV-1 envelope glycoprotein gp41 subunit, and more particularly to isolated soluble polypeptides that have an amino acid sequence that is substantially homologous to consecutive amino acids of a c-terminal portion of the membrane proximal external region (MPER) of gp41. The polypeptide can include an immunogenic epitope reactive with at least one anti-HIV antibody.

In an embodiment of the application, the polypeptide can be used in a vaccine for HIV-1. The polypeptide can have an amino acid sequence that is substantially homologous to about 50 to about 64 consecutive amino acids of SEQ ID NO: 1 and be soluble in an aqueous solution. The polypeptide can include an immunogenic epitope reactive with at least one anti-HIV antibody. The vaccine can also include a pharmaceutically acceptable carrier.

Another aspect of application relates to a DNA vaccine for HIV-1. The DNA vaccine can include a vector, a polynucleotide coupled to the vector, and a pharmaceutically acceptable carrier. The polynucleotide can have the nucleic acid sequence that encodes a polypeptide that is substantially homologous to about 50 to about 64 amino acids of SEQ ID NO: 1. The polypeptide can soluble in an aqueous solution and includes an immunogenic epitope reactive with at least one anti-HIV antibody.

### BRIEF DESCRIPTION OF THE DRAWINGS

The foregoing and other features of the application will become apparent to those skilled in the art to which the application relates upon reading the following description with reference to the accompanying drawings, in which:

FIG. 1 is a schematic diagram illustrating different polypeptides (MCON 6 (SEQ ID NO: 8), AD8 (SEQ ID NO: 9), MCON 6-1 (SEQ ID NO: 10), MCON 6-2 (SEQ ID NO: 10), MCON 6-3 (SEQ ID NO: 11)) constructs according to the application. The top four constructs are based on MCON6 sequence, whereas the bottom construct is based on HIV-1 isolate AD8. Six amino acids (including the K to Q mutation) that are different between MCON6 and AD8 are indicated above the AD8 construct. Three constructs in the middle with shorter leader sequences were generated using random hexameric oligonucleotides;

FIG. 2 is an immunoblot showing the results of SDS-PAGE analyses of various polypeptide fractions. Lanes: total cell lysate (T); soluble fraction (S); insoluble pellet fraction (P); urea solubilized fraction (U); fraction unbound to Ni-NTA (Ub); wash (W); and elutions 1 and 2 (E1 and E2). Gel was stained using Coomassie blue. The polypeptide having the amino acid sequence of SEQ ID NO: 8 is shown around the 10 kD marker;

FIG. 3 is a plot of a gel-filtration analyses of the polypeptide having the amino acid sequence of SEQ ID NO: 8. MW standards: 670, 158, 44, 17 and 1.4 kD. The polypeptide eluted with a retention time of about 8.9 minutes, which corresponds to about 98.6 kD. The analyses were done in 20 mM HEPES buffer (pH 8), and either 50 or 100 mM NaCl. Zorbax GF250 column (25 cm×0.95 cm) was used. Flow rate was 1 ml/min;

FIGS. 4A-B show circular dichroism analyses of the polypeptide having the amino acid sequence of SEQ ID NO: 8. FIG. 4A is a spectra of the polypeptide taken at various temperatures using Aviv Circular Dichroism spectrometer. The analyses were done in 5 mM HEPES buffer (pH 8), 50 mM NaCl. Polypeptide concentration was 0.3 μg/μl. After denaturing the polypeptide at 90° C., temperature was cooled back down to 50° C. and then to 10° C. and CD spectra of the polypeptide were measured again to examine efficiency of polypeptide refolding (FIG. 4B);

FIG. 5 is a graph showing immunoreactivity of the polypeptide having the amino acid sequence of SEQ ID NO: 8 to 2F5, Z13e1, and 4E10 assessed by ELISA; and

FIGS. 6A-B show resistance of the polypeptide having the amino acid sequence of SEQ ID NO: 8 to trypsin digestion. FIG. 6A is a schematic diagram of the polypeptide and potential trypsin cleavage products. FIG. 6B is an SDS-PAGE analyses of the polypeptide digested with trypsin for 10, 30, 60, 120, 180 or 240 minutes. Faint bands at 5.7 kD and 3.3 kD can also be seen.

FIG. 7 illustrates charts showing the neutralization activity of a polypeptide in accordance with the application against 4 Glade B HIV-1 isolates in three rabbits.

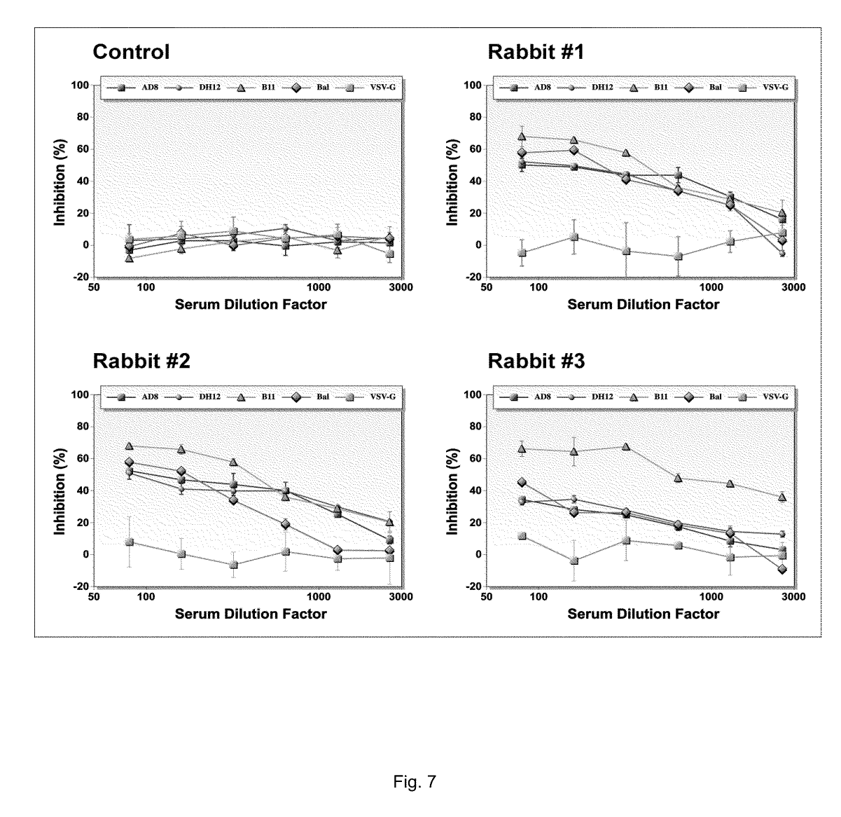

FIG. 8 illustrates HIV-1 pseudovirus neutralization assay using TZM-bl cells. Sera from control (pool of two unimmunized animals) and three immunized animals (after 4 immunizations) were tested for neutralization activity. VSV-G pseudotyped virus was used as a negative control. All viruses are Glade B, except for C7 and C33, which are from Glade C.

FIG. 9 illustrates Neutralization assay for Q23.17. Sera from three immunized rabbits (R1, R2 and R3) and mock-immunized rabbit (R4) were tested for neutralizing activity against a cladeA virus Q23.17. % neutralization is based on virus-only infections (0%). Reason for downward baseline shift with the increase in serum concentration is not known. Assays were performed in Dr David Montefiori's laboratory at Duke University.

FIG. 10 is a plot illustrating neutralizing activity of purified IgG.

FIG. 11 illustrates a graph showing the results of a peptide ELISA. Sera from immunized rabbits were analyzed by ELISA using overlapping peptides. Two peptides recognized by 2F5 are indicated by red (653 and 657).

### DETAILED DESCRIPTION

Methods involving conventional molecular biology techniques are described herein. Such techniques are generally known in the art and are described in detail in methodology treatises, such as Current Protocols in Molecular Biology, ed. Ausubel et al., Greene Publishing and Wiley-Interscience, New York, 1992 (with periodic updates). Unless otherwise defined, all technical terms used herein have the same meaning as commonly understood by one of ordinary skill in the art to which the application pertains. Commonly understood definitions of molecular biology terms can be found in, for example, Rieger et al., Glossary of Genetics: Classical and Molecular, 5th Edition, Springer-Verlag: New York, 1991, and Lewin, Genes V, Oxford University Press: New York, 1994. The definitions provided herein are to facilitate understanding of certain terms used frequently herein and are not meant to limit the scope of the application.

In the context of the application, the term “polypeptide” can refer to an oligopeptide, peptide, or protein sequence, or to a fragment, portion, or subunit of any of these, and to naturally occurring or synthetic molecules. The term “polypeptide” can also include amino acids joined to each other by peptide bonds or modified peptide bonds, i.e., peptide isosteres, and may contain any type of modified amino acids. The term “polypeptide” can also include peptides and polypeptide fragments, motifs and the like, glycosylated polypeptides, all “mimetic” and “peptidomimetic” polypeptide forms, and retro-inversion peptides (also referred to as all-D-retro or retro-enantio peptides). The term can also include linear and cyclic polypeptides.

As used herein, the term “polynucleotide” can refer to oligonucleotides, nucleotides, or to a fragment of any of these, to DNA or RNA (e.g., mRNA, rRNA, tRNA) of genomic or synthetic origin which may be single-stranded or double-stranded and may represent a sense or antisense strand, to peptide nucleic acids, or to any DNA-like or RNA-like material, natural or synthetic in origin, including, e.g., iRNA, siRNAs, microRNAs, and ribonucleoproteins. The term can also encompass nucleic acids, i.e., oligonucleotides containing known analogues of natural nucleotides, as well as nucleic acid-like structures with synthetic backbones.

As used herein, the term “antibody” can refer to whole antibodies, e.g., of any isotype (IgG, IgA, IgM, IgE, etc.), and include fragments thereof which are also specifically reactive with a target polypeptide. Antibodies can be fragmented using conventional techniques and the fragments screened for utility and/or interaction with a specific epitope of interest. Thus, the term can include segments of proteolytically-cleaved or recombinantly-prepared portions of an antibody molecule that are capable of selectively reacting with a certain polypeptide. Non-limiting examples of such proteolytic and/or recombinant fragments can include Fab, F(ab′)₂, Fab′, Fv, and single chain antibodies (scFv) containing a V[L] and/or V[H] domain joined by a peptide linker. The scFv's may be covalently or non-covalently linked to form antibodies having two or more binding sites. The term “antibody” can also include polyclonal, monoclonal, or other purified preparations of antibodies, recombinant antibodies, monovalent antibodies, and multivalent antibodies.

As used herein, the terms “effective,” “effective amount,” and “therapeutically effective amount” can refer to that amount of a composition (e.g., a vaccine) that results in amelioration of symptoms or a prolongation of survival in a subject infected with HIV (e.g., HIV-1). A therapeutically relevant effect relieves to some extent one or more symptoms of an HIV infection, or returns to normal either partially or completely one or more physiological or biochemical parameters associated with or causative of the HIV infection.

As used herein, the term “subject” can refer to any warm-blooded organism including, but not limited to, human beings, rats, mice, dogs, goats, sheep, horses, monkeys, apes, rabbits, pigs, chicken, cattle, etc.

As used herein, the term “epitope” can refer to portions of an isolated polypeptide of the application having antigenic or immunogenic activity in a subject. An “immunogenic epitope” can include a portion of an isolated polypeptide that elicits an immune response in a subject, as determined by any method known in the art. The term “antigenic epitope” can include a portion of an isolated polypeptide to which an antibody or T-cell receptor can immunospecifically bind as determined by any method well known in the art. Immunospecific binding may exclude non-specific binding, but does not necessarily exclude cross-reactivity with other antigens.

This application relates generally to isolated polypeptides that have an amino acid sequence that is substantially homologous to epitopes of the HIV-1 evelope glycoprotein gp41 subunit, and more particularly to isolated soluble polypeptides that have an amino acid sequence that is substantially homologous to consecutive amino acids of a c-terminal portion of the membrane proximal external region (MPER) of gp41. The polypeptides can include an immunogenic epitope reactive with at least one anti-HIV antibody and be soluble in an aqueous solution. The polypeptides are also structurally rigid and thermodynamically stable, include an immunogenic epitope, elicit a potent neutralizing antibody reaction by at least one anti-HIV antibody and can be expressed as soluble polypeptides without the use of a large fusion partner. The application therefore provides isolated polypeptides substantially homologous to a portion of the HIV-1 gp41 ectodomain, vaccines for HIV-1, a method for producing the soluble polypeptides, methods for inducing an immune response against HIV-1 in a subject, and a method for treating a subject having an HIV-1 infection.

In accordance with an aspect of the application, the isolated polypeptide can include about 50 to about 70 amino acids (e.g., about 50 to about 64 amino acids) and be substantially homologous to consecutive amino acids of a c-terminal portion of the MPER of gp41. By substantially homologous, it is meant the peptide has at least about 80%, about 90%, about 95%, about 96%, about 97%, about 98%, about 99% or about 100% sequence identity with consecutive amino acids of the c-terminal external region of gp41 that includes an immunogenic epitope reactive with at least one anti-HIV antibody. The immunogenic properties of the isolated polypeptide can elicit a potent, anti-HIV antibody response. For example, the immunogenic epitope can elicit a potent anti-HIV antibody response by at least one antibody selected from the group consisting of 2F5, 4E10 and Z13e1. As described in more detail below, the isolated polypeptide can be expressed as a soluble polypeptide without the use of a large fusion partner.

In an example of the application, the isolated polypeptide can include an amino acid sequence that is substantially homologous to about 50 to about 64 consecutive amino acids of SEQ ID NO: 1. SEQ ID NO: 1 is the C-terminal, 64 amino acid sequence of HIV-1 gp41 ectodomain derived from M group consensus envelope sequence. The amino acids comprising SEQ ID NO: 1 form a well-packed, three-helix bundle with a hydrophobic core. The first α-helix (W628-E648) encompasses the C-heptad repeat (HR) domain, which coils (5649-N651) into the second α-helix (5652-N669). The second helix is followed by a β-turn-like structure (W670-D674) that leads into the third α-helix with a strong curvature (I675-K683).

In another example of the application, the isolated polypeptide can include a full length polypeptide, a polypeptide fragment, a protein, or a protein fragment having the amino acid sequence that is substantially homologous to at least one of SEQ ID NO: 2 or SEQ ID NO: 3. SEQ ID NO: 2 is the C-terminal, 54 amino acid sequence of HIV-1 gp41 ectodomain derived from M group consensus envelope sequence. SEQ ID NO: 3 is the C-terminal, 54 amino acid sequence of HIV-1 gp41 ectodomain based on an authentic HIV-1 isolate (AD8). As described in more detail below, isolated polypeptides having the amino acid sequence that is substantially homologous to SEQ ID NO: 1, SEQ ID NO: 2, or SEQ ID NO: 3 can elicit broadly cross-reactive neutralizing antibody activity. Polypeptides having the amino acid sequence that is substantially homologous to SEQ ID NO: 2 or SEQ ID NO: 3, for example, may be structurally rigid, thermodynamically stable, and have an antigenically correct conformation of neutralization epitopes recognized by antibodies 2F5 and 4E10.

In another aspect of the application, an isolated polypeptide having an amino acid sequence that is substantially homologous to SEQ ID NO: 1, SEQ ID NO: 2, or SEQ ID NO: 3 can additionally comprise a leader sequence coupled to the N-terminus thereof. The leader sequence can include any polypeptide sequence capable of directing or facilitating transport of the isolated polypeptide across a cell membrane. The leader sequence can be a native human leader sequence or, alternatively, an artificially-derived leader sequence. For example, the leader sequence can comprise any one or combination of polypeptides having the amino acid sequence of SEQ ID NO: 4, SEQ ID NO: 5, SEQ ID NO: 6 or SEQ ID NO: 7.

In another aspect of the application, an isolated polypeptide having an amino acid sequence that is substantially homologous to SEQ ID NO: 1, SEQ ID NO: 2, or SEQ ID NO: 3 can additionally comprise a polyhistadine-tag. The polyhistadine-tag can include at least six histadine (His) residues coupled to either the N-terminus or C-terminus (or both) end of the isolated polypeptide. As described in more detail below, the polyhistadine-tag can be used for affinity purification of an isolated polypeptide expressed in an appropriate expression system, such as E. coli or any other prokaryotic expression system.

In an example of the application, the isolated polypeptide can have the amino acid sequence of SEQ ID NO: 1, SEQ ID NO: 2, or SEQ ID NO: 3 and additionally include a leader sequence and a polyhistadine-tag respectively coupled to the N-terminus and the C-terminus thereof. For example, the isolated polypeptide can have the amino acid sequence of SEQ ID NO: 8, SEQ ID NO: 9, SEQ ID NO: 10, SEQ ID NO: 11 or SEQ ID NO: 12.

To date, most efforts to express soluble polypeptides of gp41 have been limited to small protein fragments, including the heptad repeat (HR) regions (Root et al., Science 291:884-888, 2001), the immunodominant loop between HR1 and HR2 known as cluster I (Gnann et al., J. Virol. 61(8):2639-2641, 1987), a region between HR2 and the 2F5 epitope known as cluster II (Binley et al., AIDS Res Hum Retrovir. 2(10):911-924, 1996; Goudsmit et al., Intervirology 31(6):327-338, 1990; and Xu et al., J. Virol. 65(9):4832-4838, 1991), and the MPER (Luo et al., Vaccine 24(4):435-442, 2006; and Opalka et al., J Immunol Methods 287(1-2):49-65, 2004).

Unlike the small gp41 protein fragments of the prior art, however, the isolated polypeptides of the application are soluble in an aqueous solution. For example, an isolated polypeptide having the amino acid sequence of SEQ ID NO: 1, SEQ ID NO: 2, SEQ ID NO: 3, SEQ ID NO: 8, SEQ ID NO: 9, SEQ ID NO: 10, SEQ ID NO: 11, or SEQ ID NO: 12 can be soluble in an aqueous solution comprising a buffer or medium including, but not limited to, blood, serum, saliva, sodium chloride or saline solutions (e.g., containing about 50 mM NaCl), PBS-containing solutions (e.g., a PBS solution containing about 20 mM to about 200 mM imidazole), urea-containing solutions (e.g., containing about 1M to about 8M urea in PBS), and HEPES-containing solutions (e.g., containing about 20 mM HEPES). The aqueous solution can have a pH of between about 6 and about 10, between about 7 and about 8, or about physiological pH (7.4).

In another aspect of the application, a method is provided for producing a soluble polypeptide comprising a portion of the HIV-1 gp41 ectodomain. Unlike prior art methods for producing a soluble polypeptide comprising a portion of the HIV-1 gp41 ectodomain, the method of the application provides a technique for expressing soluble polypeptides comprising a portion of the HIV-1 gp41 ectodomain without the need for a large fusion partner. As described in more detail below, the fact that a large fusion partner is not needed is significant from a vaccine development perspective as large fusion partners can diminish antibody responses against epitopes on target immunogens.

One step of the method can include transforming a host cell with a vector comprising an isolated polynucleotide having the nucleic acid sequence of SEQ ID NO: 13, SEQ ID NO: 14, SEQ ID NO: 15, SEQ ID NO: 16, SEQ ID NO: 17, SEQ ID NO: 18, SEQ ID NO: 19 or SEQ ID NO: 20. The vector can include any vector that will allow an isolated polynucleotide (e.g., having SEQ ID NO: 16) within a host cell. For example, the vector can comprise a plasmid, promoter, and polyadenylation/transcriptional termination sequence arranged in the correct order to obtain expression of the isolated polynucleotide. Other examples of vectors are known in the art and described below.

In an example of the method, an isolated polynucleotide having the nucleic acid sequence of SEQ ID NO: 14 can be inserted into a DNA plasmid vector, such as a pET-21(a) vector (NOVAGEN) after PCR-amplification of the isolated polynucleotide using a pair of primers with appropriate restriction sites (e.g., BamHI and EcoRI sites). For example, a sense primer having the nucleic acid sequence of SEQ ID NO: 21 and an anti-sense primer having the nucleic acid sequence of SEQ ID NO: 22 can be used to PCTR-amplify the isolated polynucleotide. Additional modifications to the isolated polynucleotide, such as a base pair modification resulting in a 54K→54Q mutation can be made just upstream of the polyhistadine-tag to remove a trypsin digest site, for example.

After constructing an appropriate expression vector, the vector can be transformed into a host cell. In an example of the method, the recombinant pET-21(a) plasmid (described above) can be transformed into E. coli BL21(DE3)pLysS (INVITROGEN) and then cultured overnight at about 28° C. in superbroth containing about 50 μg/ml of Ampicillin. Next, the culture can be seeded into fresh superbroth (e.g., using about a 1/50 dilution) and cultured to an OD600 of about 0.8 at about 24° C. Expression of the polypeptide (e.g., SEQ ID NO: 8) corresponding to the isolated polynucleotide, as well as the pET leader sequence and polyhistadine-tag can then be induced with about 1 mM IPTG for about 5 hours.

After induction of polypeptide expression, the expressed polypeptide can be collected and purified. Various methods for purifying polypeptides are known in the art and include, for example, ultracentrifugation and chromatographic techniques, such as affinity column chromatography. In an example of the method, the E. coli host cells can be lysed in PBS by sonication, followed by centrifugation at about 10,000 rpm for about 20 minutes to pellet inclusion bodies containing the expressed polypeptide. The resultant pellet can then be solubilized with PBS containing about 8M urea. Next, solubilized polypeptides can be purified using the Ni-NTA Purification System (QIAGEN), for example.

The isolated polypeptides can then be slowly renatured through sequential incubations with 10 bed volumes of PBS containing about 8M urea, about 6M urea, about 4M urea, about 3M urea, about 2M urea, about 1M urea, and without urea. Polypeptides bound to Ni-NTA resin can then be washed with PBS containing about 20 mM imidazole and eluted with PBS containing about 200 mM imidazole. Eluted polypeptides can be dialyzed against PBS (at a pH of about 7.4) or about 20 mM HEPES (at about a pH of about 8) with or without about 50 mM NaCl. Polypeptide concentration can then be determined using a known assay, such as a Bradford assay. As described in more detail below, the purified polypeptide can be used to form a therapeutic composition, such as a vaccine or pharmaceutical composition.

Another aspect of the application can include a vaccine for HIV (e.g., HIV-1). The vaccine can comprise an isolated polypeptide including a portion of the HIV-1 gp41 ectodomain and a pharmaceutically acceptable carrier. For example, the isolated polypeptide comprising the vaccine can have the amino acid sequence that is substantially homologous to SEQ ID NO: 1, SEQ ID NO: 2, SEQ ID NO: 3, SEQ ID NO: 8, SEQ ID NO: 9, SEQ ID NO: 10, SEQ ID NO: 11 or SEQ ID NO: 12. More particularly, the vaccine can comprise an isolated polypeptide having the amino acid sequence of SEQ ID NO: 8 and a pharmaceutically acceptable carrier.

In another aspect of the application, the vaccine can comprise a pharmaceutically acceptable carrier and an isolated polynucleotide having the nucleic acid sequence of SEQ ID NO: 13, SEQ ID NO: 14, SEQ ID NO: 15, SEQ ID NO: 16, SEQ ID NO: 17, SEQ ID NO: 18, SEQ ID NO: 19 or SEQ ID NO: 20. For example, the vaccine can comprise an isolated polynucleotide having the nucleic acid sequence of SEQ ID NO: 16 and a pharmaceutically acceptable carrier.

While it is possible that the vaccine can comprise an isolated polypeptide (e.g., having the amino acid sequence of SEQ ID NO: 8) or an isolated polynucleotide (e.g., having the nucleic acid sequence of SEQ ID NO: 16) in a pure or substantially pure form, it will be appreciated that the vaccine can additionally or optionally include the isolated polypeptide or polynucleotide and a pharmaceutically acceptable carrier or other therapeutic agent. For example, the pharmaceutically acceptable carrier can include a physiologically acceptable diluent, such as sterile water or sterile isotonic saline. As used herein, the term “pharmaceutically acceptable carrier” can refer to any and all solvents, dispersion media, coatings, antibacterial and antifungal agents, isotonic and absorption delaying agents, and the like.

Additional components that may be present with the vaccine can include adjuvants, preservatives, chemical stabilizers, and/or other proteins. Typically, stabilizers, adjuvants, and preservatives are optimized to determine the best formulation for efficacy in a subject. Exemplary preservatives can include, but are not limited to, chlorobutanol, potassium sorbate, sorbic acid, sulfur dioxide, propyl gallate, the parabens, ethyl vanillin, glycerin, phenol, and parachlorophenol. Suitable stabilizing ingredients can include, for example, casamino acids, sucrose, gelatin, phenol red, N-Z amine, monopotassium diphosphate, lactose, lactalbumin hydrolysate, and dried milk. Other examples of pharmaceutically acceptable carriers are known in the art and described below.

A vaccine comprising an isolated polypeptide (e.g., having the amino acid sequence of SEQ ID NO: 8) or isolated polynucleotide (e.g., having the nucleic acid sequence of SEQ ID NO: 16) can be used either prophylactically or therapeutically. When provided prophylactically, the vaccine can be provided in advance of any evidence of an active HIV infection and thereby attenuate or prevent HIV infection. For example, a human at high risk for HIV infection can be prophylactically treated with a vaccine comprising an isolated polypeptide having the amino acid sequence of SEQ ID NO: 8 and a pharmaceutically acceptable carrier. When provided therapeutically, the vaccine can be used to enhance a subject's own immune response to the antigens present as a result of HIV infection. The vaccine, which can act as an immunogen, may be a partially or substantially purified polypeptide or polynucleotide. It will be appreciated that an isolated polypeptide or polynucleotide comprising the vaccine can be conjugated with one or more lipoproteins, administered in liposomal form, or with an adjuvant.

Another aspect of the application can include a method for inducing an immune response against HIV or an HIV epitope in a subject. The method can include administering to the subject an effective amount of an isolated polypeptide (e.g., having the amino acid sequence of SEQ ID NO: 8), an isolated polynucleotide (e.g., having the nucleic acid sequence of SEQ ID NO: 16), a vector including the isolated polynucleotide, a host cell including the vector, or some other pharmaceutical composition, such as a vaccine that elicits the immune response and thereby prevents or inhibits HIV infection in the subject.

Inhibiting a viral infection can refer to inhibiting the onset of a viral infection, inhibiting an increase in an existing viral infection, or reducing the severity of the viral infection. In this regard, one of ordinary skill in the art will appreciate that while complete inhibition of the onset of a viral infection is desirable, any degree of inhibition of the onset of a viral infection is beneficial. Likewise, one of ordinary skill in the art will appreciate that while elimination of viral infection is desirable, any degree of inhibition of an increase in an existing viral infection or any degree of a reduction of a viral infection is beneficial.

Inhibition of a viral infection can be assayed by methods known in the art, such as by assessing viral load. Viral loads can be measured by methods known in the art, such as by using PCR to detect the presence of viral nucleic acids or antibody-based assays to detect the presence of viral protein in a sample (e.g., blood) from a subject. Alternatively, the number of CD4+ T cells in a viral-infected subject can be measured. A treatment that inhibits an initial or further decrease in CD4+ T cells in a viral-infected subject, or that results in an increase in the number of CD4+ T cells in a viral-infected subject, for example, may be considered an efficacious or therapeutic treatment.

As noted above, one aspect of the application can comprise administering to a subject a vector including an isolated polynucleotide (e.g., having the nucleic acid sequence of SEQ ID NO: 16). Vectors can include polynucleotide vectors, such as naked DNA or plasmids and viral vectors, such as retroviral vectors, parvovirus-based vectors (e.g., adenoviral-based vectors and adeno-associated virus (AAV)-based vectors), lentiviral vectors (e.g., Herpes simplex (HSV)-based vectors), and hybrid or chimeric viral vectors, such as an adenoviral backbone with lentiviral components or an adenoviral backbone with AAV components. Other examples of vectors, as well as methods for vector construction are known in the art.

The vector can additionally or optionally comprise any suitable promoter and/or other regulatory sequence(s) (e.g., transcription and translation initiation and termination codon specific to the type of host cell) to control the expression of an isolated polynucleotide (e.g., having the nucleic acid sequence of SEQ ID NO: 16). The promoter can be a native or normative promoter operably linked to the isolated polynucleotide. The selection of promoters, including various constitutive and regulatable promoters is within the skill of an ordinary artisan. Examples of regulatable promoters can include inducible, repressible, and tissue-specific promoters. Specific examples of promoters can include viral promoters, such as adenoviral promoters and AAV promoters. Combining a polynucleotide with a promoter is within the skill of one in the art.

Another aspect of the application can include administering to a subject at least one host cell comprising an isolated polypeptide (e.g., having the amino acid sequence of SEQ ID NO: 8) or an isolated polynucleotide (e.g., having the nucleic acid sequence of SEQ ID NO: 16), optionally in the form of a vector. Any host cell can be used including, for example, E. coli (e.g., E. coli Tb-1, TG-1, DH5α, XL-Blue MRF′, SA2821, and Y1090), Bacillus subtilis, Salmonella typhimurium, Serratia marcescens, Pseudomonas (e.g., P. aerugenosa), N. grassa, insect cells (e.g., Sf9, Ea4), yeast cells (e.g., S. cerevisiae), and cells derived from a mammal, including human cell lines. Specific examples of eukaryotic host cells can include VERO, HeLa, 3T3, Chinese hamster ovary (CHO) cells, W138 BHK, COS-7, and MDCK cells. Methods of introducing vectors into isolated host cells, as well as the culture and selection of transformed host cells in vitro are known in the art and can include, for example, the use of calcium chloride-mediated transformation, transduction, conjugation, triparental mating, DEAE, dextran-mediated transfection, infection, membrane fusion with liposomes, high velocity bombardment with DNA-coated microprojectiles, direct microinjection into single cells, and electroporation.

An isolated polypeptide (e.g., having the amino acid sequence of SEQ ID NO: 8), an isolated polynucleotides (e.g., having the nucleic acid sequence of SEQ ID NO: 16), a vector, a host cell, and/or a vaccine can be administered to a subject alone or in combination with a pharmaceutically acceptable carrier. By “pharmaceutically acceptable”, it is meant a material which is not biologically or otherwise undesirable (i.e., the material can be administered to a subject without causing any undesirable biological effects or interacting in a deleterious manner with any of the other components of the pharmaceutical composition). The carrier can be selected to minimize any degradation of the isolated polypeptide, the isolated polynucleotide, the vector, the host cell, and/or the vaccine to minimize any adverse side effects in the subject.

Pharmaceutical carriers and their formulations are described in Remington: The Science and Practice of Pharmacy (19th ed.) ed. A. R. Gennaro, Mack Publishing Company, Easton, Pa. (1995). Pharmaceutical carriers can include, for example, sterile water, saline, Ringer's solution, dextrose solution, and buffered solutions at physiological pH. An appropriate amount of a pharmaceutically acceptable salt can be used in the formulation to render the formulation isotonic. For example, the pH of the solution can be from about 5 to about 8, or from about 7 to about 8. Additional or optional carriers can include sustained-release preparations, such as semipermeable matrices of solid hydrophobic polymers (e.g., in the form of films, liposomes, or microparticles) that contain isolated polypeptides. It will be appreciated that certain carriers may be more or less appropriate depending upon, for example, the route of administration and concentration of the composition being administered.

Compositions comprising an isolated polypeptide (e.g., having the amino acid sequence of SEQ ID NO: 8) or an isolated polynucleotide (e.g., having the amino acid sequence of SEQ ID NO: 16) can additionally or optionally include carriers, thickeners, diluents, buffers, preservatives, surface agents, and the like. Additionally, compositions can include one or more active agents, such as antimicrobial agents, anti-inflammatory agents, anesthetics, anti-viral agents, and the like. For example, active agents can include azidothymidine (AZT), Cyclosporin A, inactivated virus, interleukin (IL)-2, IL-12, CD40 ligand and IL-12, IL-7, interferons, and HIV antibodies.

Compositions comprising an isolated polypeptide (e.g., having the amino acid sequence of SEQ ID NO: 8) or an isolated polynucleotide (e.g., having the nucleic acid sequence of SEQ ID NO: 16) can be administered in any suitable manner, depending on the area to be treated and whether local or systemic treatment is desired. Administration can be topically (e.g., ophthalmically, vaginally, rectally, intranasally, transdermally, etc.), orally, by inhalation, or parenterally (e.g., by intravenous drip or by subcutaneous, intracavity, intraperitoneal, or intramuscular injection). Topical intranasal administration can include delivery of a composition into the nose and nasal passages through one or both of the nares. Nasal delivery can be accomplished by aerosolization of the composition (e.g., a spraying mechanism or droplet mechanism). Delivery can also be made directly to any area of the respiratory system (e.g., lungs) via intubation.

If the composition is to be administered parenterally, the administration is generally by injection. Injectable compositions can be prepared in conventional forms, either as liquid solutions or suspensions, solid forms suitable for suspension in liquid prior to injection, or as emulsions. Additionally, parenteal administration can involve the preparation of a slow-release or sustained-release system to maintain a constant dosage. Preparations for parenteral administration can include sterile aqueous or non-aqueous solutions, suspensions, and emulsions. Examples of non-aqueous solvents can include propylene glycol, polyethylene glycol, vegetable oils, such as olive oil, and injectable organic esters, such as ethyl oleate. Aqueous carriers can include water, alcoholic/aqueous solutions, emulsions or suspensions, including saline and buffered media. Parenteral vehicles can include sodium chloride solutions, Ringer's dextrose, dextrose, sodium chloride, lactated Ringer's, or fixed oils. Intravenous vehicles can include fluid and nutrient replenishers, electrolyte replenishers (such as those based on Ringer's dextrose), and the like. Preservatives and other additives, such as antimicrobials, anti-oxidants, chelating agents, inert gases, and the like can also be present.

It will be appreciated that some compositions can be administered as a pharmaceutically acceptable acid- or base-addition salt formed by reaction with inorganic acids, such as hydrochloric acid, hydrobromic acid, perchloric acid, nitric acid, thiocyanic acid, sulfuric acid, and phosphoric acid, and organic acids, such as formic acid, acetic acid, propionic acid, glycolic acid, lactic acid, pyruvic acid, oxalic acid, malonic acid, succinic acid, maleic acid, and fumaric acid, or by reaction with an inorganic base, such as sodium hydroxide, ammonium hydroxide, potassium hydroxide, and organic bases, such as mono-, di-, trialkyl, and aryl amines and substituted ethanolamines.

It will also be appreciated that probiotic therapies may also be used to induce an immune response against HIV. For example, viable host cells containing an isolated polynucleotide (e.g., having the nucleic acid sequence of SEQ ID NO: 16) or a vector expressing the corresponding polypeptide (e.g., having the amino acid sequence of SEQ ID NO: 8) can be used to deliver the polypeptide in vivo. For example, viable prokaryotic host cells, such as lactobacilli, enterococci, or other common bacteria (e.g., E. coli) can be used. Such host cells can be suitably transformed using the isolated polynucleotide (or a vector comprising the isolated polynucleotide), and then administered to the subject where expression of the corresponding polypeptide can occur and thereby induce an immune response against HIV.

If ex vivo methods are employed, cells or tissues can be removed and maintained outside the body of a subject according to standard protocols known in the art. For example, an isolated polynucleotide having the nucleic acid sequence of SEQ ID NO: 16 can be introduced into the cells via any gene transfer mechanism, such as calcium phosphate-mediated gene delivery, electroporation, microinjection, or proteoliposomes. The transduced cells then can then be infused (e.g., with a pharmaceutically acceptable carrier) or homotopically transplanted back into the subject per standard methods for the cell or tissue type. Standard methods are known in the art for transplantation or infusion of cells into a subject.

The exact amount of the composition required to treat an HIV infection will vary from subject to subject, depending on the species, age, gender, weight, and general condition of the subject, the nature of the virus, the existence and extent of viral infection, the particular isolated polypeptide (e.g., having the amino acid sequence of SEQ ID NO: 8) or isolated polynucleotide (e.g., having the nucleic acid sequence of SEQ ID NO: 16) used, the route of administration, and whether other drugs are included in the regimen. An appropriate amount can be determined, however, by one of ordinary skill in the art using routine experimentation. The dosage ranges for administration of the compositions can include those large enough to produce the desired effect; however, the dosage should not be so large as to cause adverse side effects, such as unwanted cross-reactions, anaphylactic reactions, and the like. Dosage can vary, and can be administered in one or more (e.g., two or more, three or more, four or more, or five or more) doses daily, for one or more days. The composition can be administered before viral infection or immediately upon determination of viral infection, and then continuously administered until the virus is undetectable.

Effective dosages and schedules for administering compositions may be determined empirically, and making such determinations is routine to one of ordinary skill in the art. The skilled artisan will appreciate that dosage(s) of the composition will vary depending upon, for example, the species of the subject, the route of administration, other drugs being administered, and the age, condition, sex, and extent of the disease in the subject.

The following example is for the purpose of illustration only and is not intended to limit the scope of the claims, which are appended hereto.

### Example 1

### Cloning

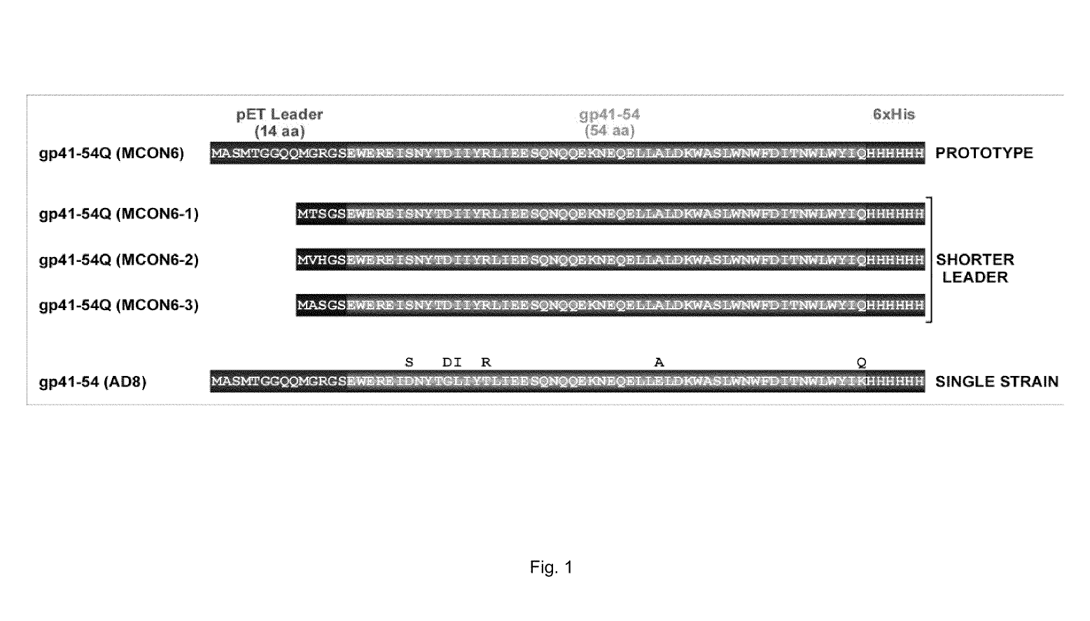

The isolated polypeptide having the amino acid sequence of SEQ ID NO: 8 was constructed by cloning the C-terminal 54 amino acids of gp41 ectodomain from M group consensus sequence into pET-21(a) vector (NOVAGEN). The polynucleotide having the nucleic acid sequence of SEQ ID NO: 2 was PCR-amplified using a pair of primers with appropriate restriction sites (sense: SEQ ID NO: 21; anti-sense: SEQ ID NO: 22). An inconsequential Lys (K) to Gln (O) mutation was introduced just upstream of the 6×His tag to remove a trypsin digest site (eliminating this trypsin site would allow removal, if necessary, of the leader sequence while preventing cleavage of the 6×His tag needed for protein purification). As shown in FIG. 1, the isolated polypeptide having the amino acid sequence of SEQ ID NO: 8 contains a 14 amino acid sequence from the vector, followed by 54 amino acids of gp41 ectodomain and a 6×His tag.

### Protein Expression and Purification

E. coli BL21(DE3)pLysS (INVITROGEN) was transformed with the recombinant plasmid (above) and cultured overnight at 28° C. in superbroth containing 50 μg/ml of Ampicillin. Culture was seeded into fresh superbroth (1:50 dilution) and cultured to OD₆₀₀ of 0.8 at 24° C. Protein expression was induced with 1 mM IPTG for 5 hours.

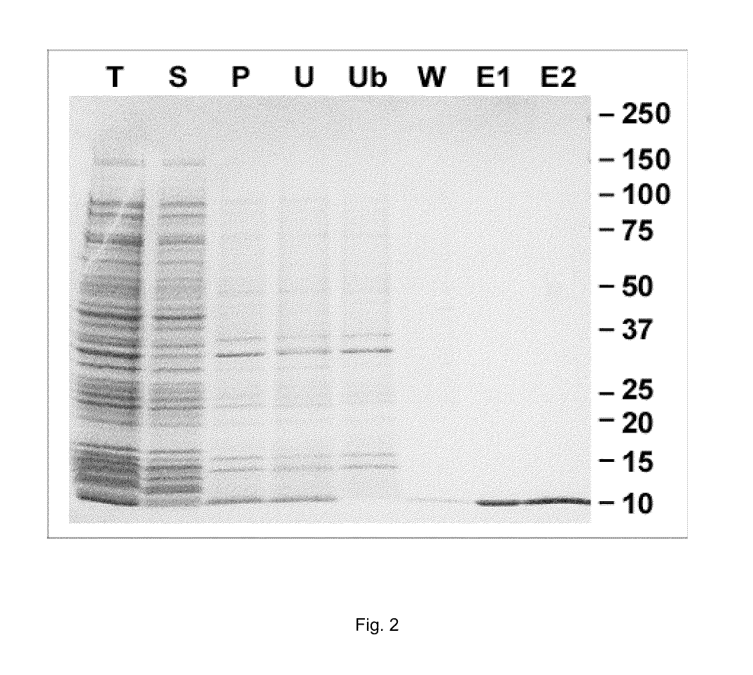

To purify the expressed polypeptide having SEQ ID NO: 8, E. coli were lysed in PBS by sonication. Inclusion bodies containing the polypeptide were pelleted by centrifugation at 10,000 rpm for 20 minutes in a SORVALL Superspeed Centrifuge. The pellet was solubilized with PBS containing 8M urea. Solubilized polypeptides were bound to Ni-NTA resin (QIAGEN). The polypeptide was slowly renatured through sequential incubations with 10 bed volumes of PBS containing 8M urea, 6M urea, 4M urea, 3M urea, 2M urea, 1M urea, and without urea. Polypeptides bound to Ni-NTA resin were washed with PBS containing 20 mM imidazole and then eluted with PBS containing 200 mM imidazole. The eluted polypeptides were dialyzed against PBS (pH 7.4) or 20 mM HEPES (pH 8.0) with or without 50 mM NaCl. Polypeptide concentrations were determined by Bradford assays. A typical yield for the polypeptide is about 4-5 mg/liter of cell culture. Purification of the polypeptide is shown in FIG. 2.

### Characterization of Biochemical Properties

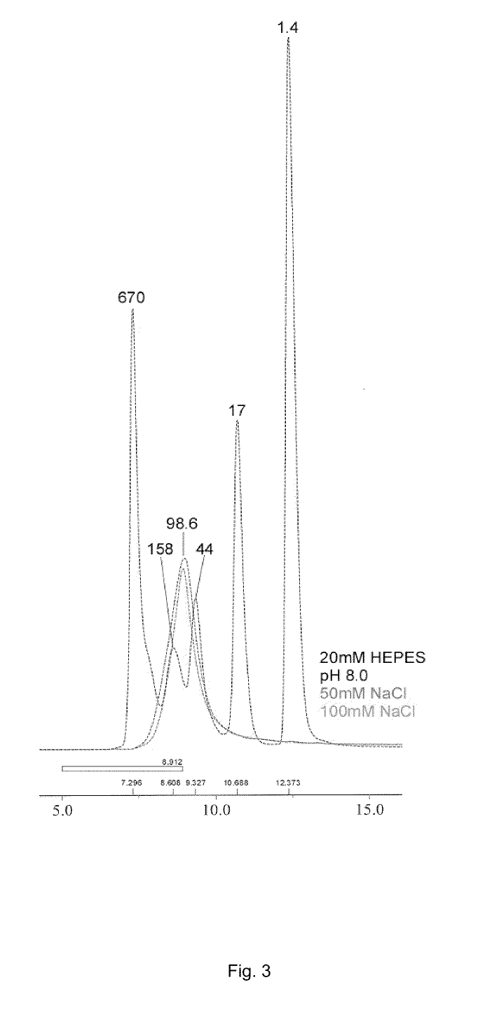

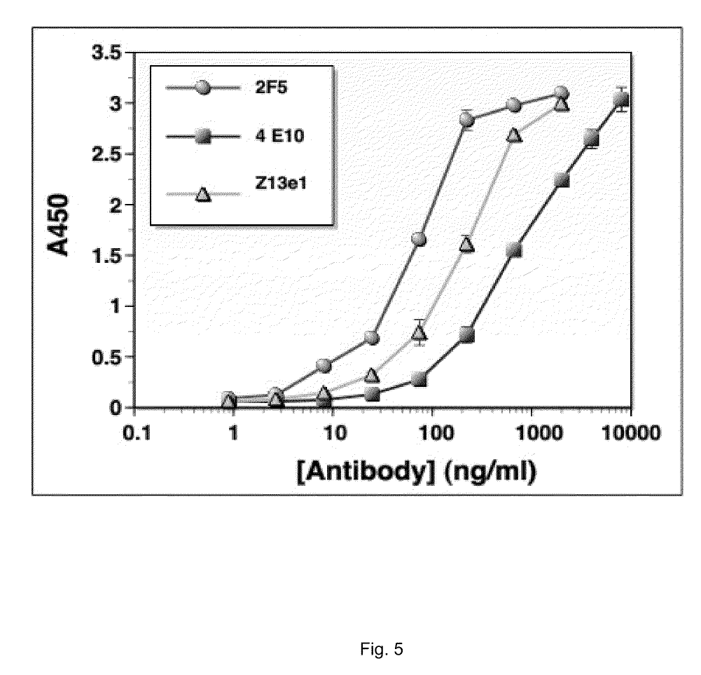

The theoretical molecular weight of the polypeptide having SEQ ID NO: 8 is about 9.1 kD. However, when the purified polypeptide was subjected to gel-filtration analyses, the polypeptide migrated with an empirical molecular weight of about 98.6 kD (FIG. 3). The result indicates that the polypeptide likely forms a multimer. Regardless, the polypeptide is able to present 2F5 and 4E10 neutralization epitopes (FIG. 5).

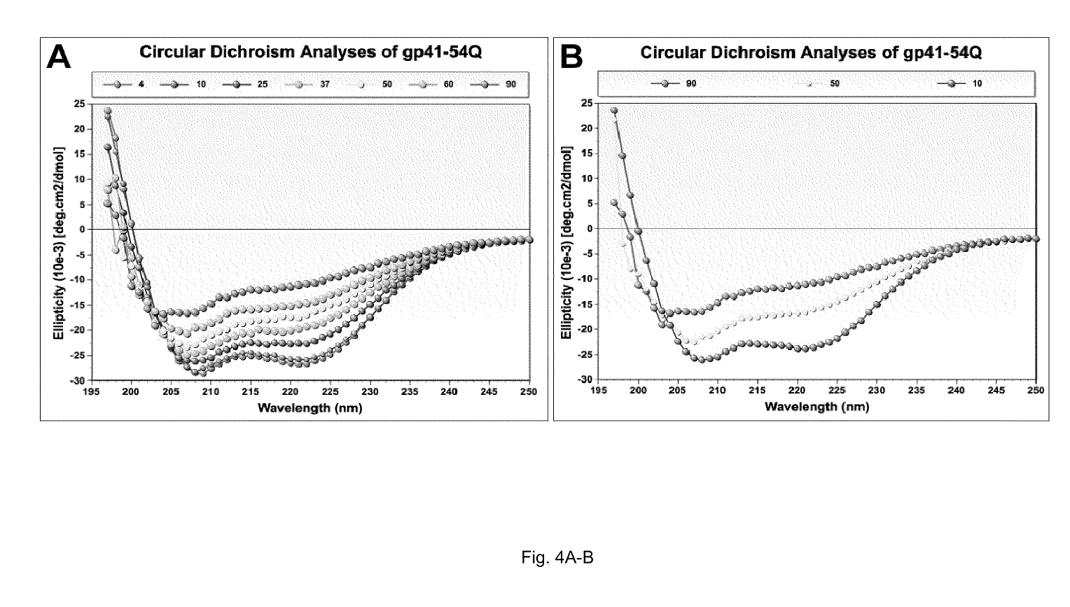

To examine secondary structure of the polypeptide, purified polypeptide was analyzed by circular dichroism spectrometer. As shown in FIG. 4, the polypeptide has highly ordered secondary structure, mostly composed of α-helices. The structure is highly thermostable, losing its α-helicity only when temperature reached 90° C. (FIG. 4A). The polypeptide returned to its original structure when the temperature was cooled down to 50° C. and 10° C. (FIG. 4B).

### Characterization of Antigenic Properties

To confirm that the polypeptide maintains antigenically correct epitope conformation, the polypeptide was probed with 2F5, 4E10 and Z13e1. As shown in FIG. 5, the polypeptide was efficiently recognized by all three antibodies.

### Variations of Second Generation Antigen

Three variants of the polypeptide having SEQ ID NO: 8 were generated, each with different five amino acid leader sequences (FIG. 1). To demonstrate that the properties of the polypeptide having SEQ ID NO: 8 were not dependent on the particular amino acid sequence or the K to Q mutation, we generated another polypeptide (SEQ ID NO: 9) based on an authentic HIV-1 isolate AD8 (FIG. 1). Gel-filtration analyses showed that the polypeptide behaved similar to the polypeptide having SEQ ID NO: 8.

### Trypsin Treatment

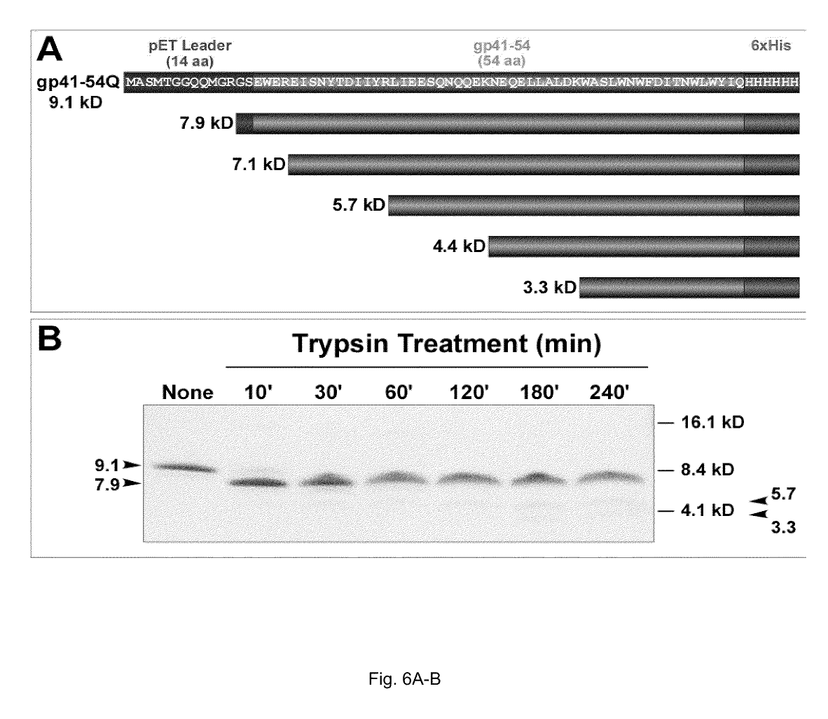

To examine whether the polypeptide having SEQ ID NO: 8 is also resistant to trypsin digestion, purified polypeptide was treated with trypsin for various times. As shown in FIG. 6A, there are five potential cleavage sites. As shown in FIG. 6B, however, the only site susceptible to trypsin digestion was the first Arg (R) residue within the leader sequence, resulting in a 7.9 kD fragment. The 7.9 kD fragment was resistant to trypsin even after 4 hours. This result indicates that the polypeptide forms a highly ordered secondary structure that is resistant to trypsin digestion.

### Example 2

### Antigen Preparation

In order to remove the leader sequence in pET construct (MASMTGGQQMGR) (SEQ ID NO: 23), 10 mg of purified gp41-54Q was mixed with 50 μg of trypsin in 20 mM HEPES buffer (pH8.0) with 50 mM NaCl, and incubated at 37° C. for 15 min. After incubation, insoluble fraction was removed by centrifugation at 12000 rpm for 10 min. 2 ml of Ni-NTA resin, equilibrated with the same buffer, was added into the soluble fraction, and rocked at 4° C. for 2 hr. Resin was loaded onto a column, and unbound fraction was removed. Resin was washed with 10 bed-volume of PBS containing 20 mM immidazole, and then bound proteins were eluted with PBS containing 200 mM immidazole. Eluted protein (gp41-54Q(T)) was dialyzed with PBS. Subsequently, insoluble protein was removed by centrifugation.

reparation of Adjuvant (Zinc-Chitosan)

Zinc-chitosan was prepared as previously reported (Seferian, P. G., and M. L. Martinez. 2000). Immune stimulating activity of two new chitosan containing adjuvant formulations. Vaccine 19:661-8 Briefly, 2 g of chitosan was dissolved in 100 ml of 2% acetic acid and sterilized by autoclaving. The 2% chitosan solution was diluted 1:1 using deionized water and 4 ml of the resulting 1% chitosan solution was added to 10 ml of 0.2 M zinc acetate.

The resulting suspension was mixed on a rocker for 4 h at room temperature. The mixture was then sonicated using a Branson Digital Sonicator for 5 min and the pH was adjusted to 12.0-12.5 with 10 M NaOH during sonication. This results in a fine white precipitate, which was pelleted by centrifugation at 1000×g for 10 min. The pellet was washed twice with PBS, pH 7.2 by centrifugation at 1000×g for 10 min.

Loading Antigen onto Zinc-Chitosan

To optimize antigen binding to zinc-chitosan, gp41-54Q(T) was placed in PBS, pH 8. Prior to adding antigen, zinc-chitosan was washed twice with PBS (pH 8) by centrifugation at 1000×g for 10 min. For each 200 μg of gp41-54Q(T) to be injected, 200 mg of zinc-chitosan was used. The suspension was continuously mixed for 3 h at room temperature. The adjuvant-antigen complex was pelleted by centrifugation at 1000×g for 10 min. The pellet was resuspended in 800 μl of PBS (pH 7.4).

### Immunization

Female New Zealand white rabbits a (about 3 kg) were used. Rabbits were anesthetized with acepromazine and injection sites were shaved. Three animals were immunized. Each rabbit was immunized with 200 μg of gp41-54Q(T) in 800 μl of adjuvant-antigen complex subcutaneously (7-8 sites on either left or the right sized of the back). Two control animals were injected with zinc-chitosan only without the antigen. Rabbits were boosted 4 and 9 weeks after the initial immunization. Blood samples were collected two weeks after each immunization.

### Neutralization Assay

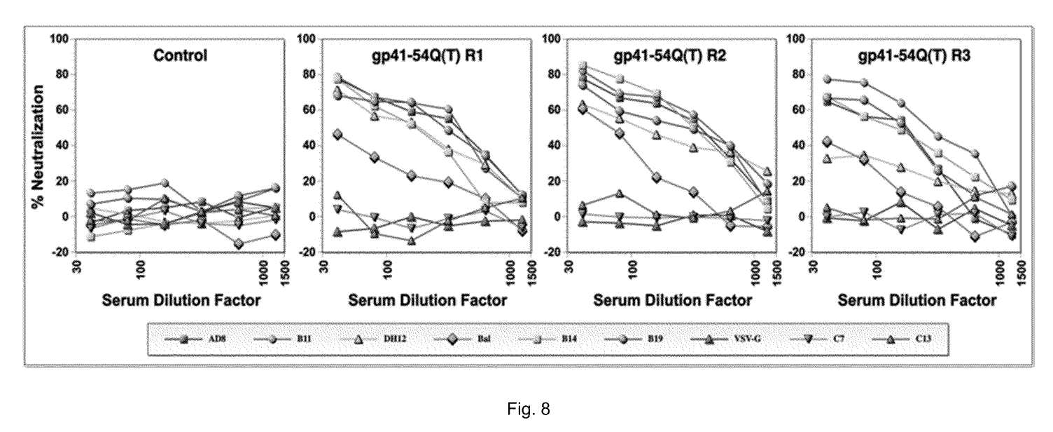

Single round infection assays in TZM-bl cells using pseudoviruses were performed as previously described (Penn-Nicholson, A., D. P. Han, S. J. Kim, H. Park, R. Ansari, D. C. Montefiori, and M. W. Cho. 2008. Assessment of antibody responses against gp41 in HIV-1-infected patients using soluble gp41 fusion proteins and peptides derived from M group consensus envelope. Virology 372:442-56). To date, neutralization activity was tested against 4 Glade B HIV-1 isolates (AD8, DH12, B11 and Bal). VSV-G pseudovirus was used as a negative control. As shown in FIG. 8, serum from all three rabbits exhibited neutralizing activity against HIV-1 pseudoviruses, but not against VSV-G pseudovirus. As expected, serum from the control animals (pooled from two animals) did not exhibit any neutralizing activity. These results demonstrate that our antigen can elicit neutralizing antibodies against HIV-1.

Evaluation of Immunogenic Properties of gp41-MPER-Based Antigens

Three female New Zealand white rabbits were immunized subcutaneously with 200 μg of gp41-54Q four times on weeks 0, 4, 9 and 15. Two control animals were immunized with a preparation containing Zn-chitosan only. Serum samples were collected two weeks after each immunization and evaluated by ELISA and HIV-1 pseudovirus neutralization assay. High titers of antibodies were elicited in immunized animals after four immunizations (end-point titers of 2×10⁶, 6×10⁶, and 2×10⁷ in rabbits 1, 2 and 3, respectively). More importantly, quite potent HIV-specific neutralizing activity was observed with significant breadth, albeit two Glade C viruses we tested were not susceptible (FIG. 8). VSV-G-pseudotyped virus was not neutralized, demonstrating specificity.

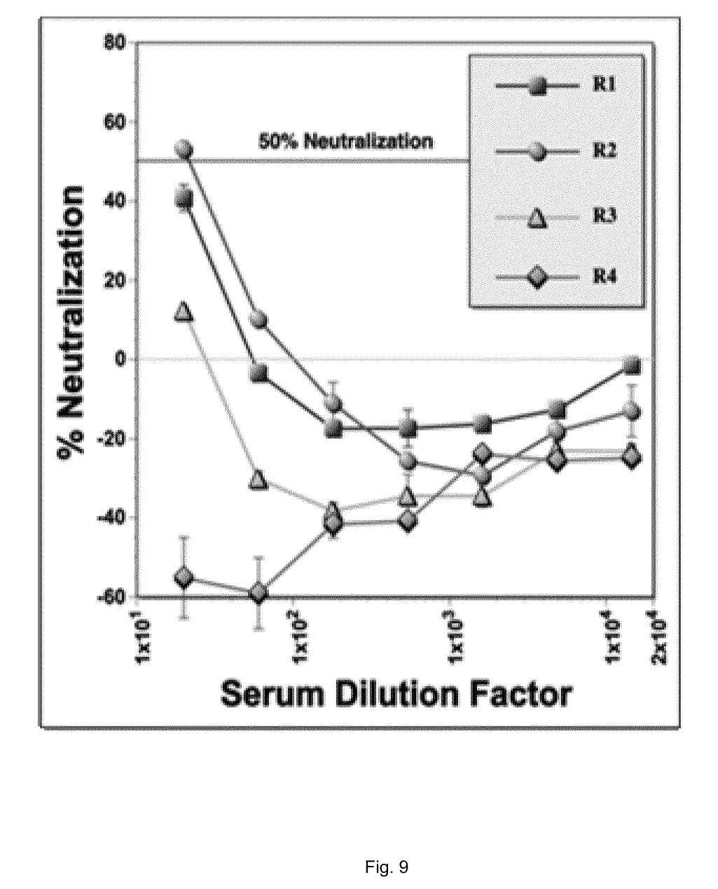

To confirm our results, serum samples were independent verified. As shown in Table 1, antisera from three immunized rabbits (R1, R2 and R3) were highly effective against Tier 1A and 1B viruses from clades B and C. Neutralizing activity against two Tier 2 viruses was also detected for Rabbit #2 (one Glade B and one Glade A). Serum from mock-immunized rabbit (R4) did not exhibit any detectable neutralizing activity. Rabbit #2 exhibited the most potent neutralizing activity, consistent with results from our laboratory. It is noteworthy that sera from all three immunized rabbits neutralized two Glade C viruses which were not neutralized by 2F5 (MW965.26 and 92BR025.9). More importantly, although sera from immunized rabbits were not able to reach 50% neutralization at 1:20 dilution for most of Tier 2 viruses, significant neutralizing activities were detected against most viruses. Neutralization of viruses that fall into this category are shaded in tan in the table. As an example, neutralization assay for cladeA virus Q23.17 is shown in FIG. 9.

TABLE C1
Neutralization assay (Conducted by Dr. David Montefiori's laboratory

| | | | | ID50 in TZM-b1 cells¹ | | | | |
|---|---|---|---|---|---|---|---|---|
| Virus | Stock ID # | Clade | Tier | R1 | R2 | R3 | R4 | 2F5 |
| SF162.LS | 2193 | B | 1A | 838 | 1262 | 370 | <20 | 0.5 |
| W61D-TCLA.71 | 983 | B | 1A | 986 | 1751 | 658 | <20 | <0.1 |
| MN | 1127 | B | 1A | 1635 | 3092 | 1005 | <20 | <0.01 |
| Bal.26 | 723 | B | 1B | 86 | 135 | 47 | <20 | 1.1 |
| BZ167.12 | 1121 | B | 1B | 29 | 43 | <20 | <20 | 2.2 |
| Bx08 | 1124 | B | 1B | 72 | 107 | 41 | <20 | 1.1 |
| SS1196.1 | 2436 | B | 1B | 47 | 88 | 26 | <20 | 18.0 |
| MW965.26 | 2842 | C | 1A | 1329 | 2025 | 650 | <20 | >25 |
| 92BR025.9 | 1180 | C | 1B | 56 | 105 | 34 | <20 | >25 |
| 6535.3 | 1776 | B | 2 | <20 | <20 | <20 | <20 | 1.8 |
| QH0692.42 | 2190 | B | 2 | <20 | <20 | <20 | <20 | 0.3 |
| SC42261.8 | 951 | B | 2 | <20 | <20 | <20 | <20 | 0.2 |
| PVO.4 | 845 | B | 2 | <20 | 28 | <20 | <20 | >25 |
| Du156.12 | 2154 | C | 2 | <20 | <20 | <20 | <20 | >25 |
| Du422.1 | 1340 | C | 2 | <20 | <20 | <20 | <20 | >25 |
| ZM197M.PB7 | 2538 | C | 2 | <20 | <20 | <20 | <20 | >25 |
| CAP45.2.00.G3 | 2365 | C | 2 | <20 | <20 | <20 | <20 | >25 |
| Q23.17 | 1659 | A | 2 | <20 | 22 | <20 | <20 | 2.0 |
| Q461,e2 | 2882 | A | 2 | <20 | <20 | <20 | <20 | 4.1 |
| Q769.d22 | 2661 | A | 2 | <20 | <20 | <20 | <20 | 1.3 |
| Q842.d12 | 1607 | A | 2 | <20 | <20 | <20 | <20 | 7.7 |

¹Values are the sample dilution at which relative luminescence units (RLUs) were reduced 50% compared to virus control wells (no test sample). Positive neutralization is shown in boldface type. Values for 2F5 are μg/ml.

These results demonstrate that our gp41-54Q antigen can elicit fairly broad neutralizing antibodies against HIV-1, albeit weak against some virus isolates.

### Demonstration of Antibody-Mediated Neutralization

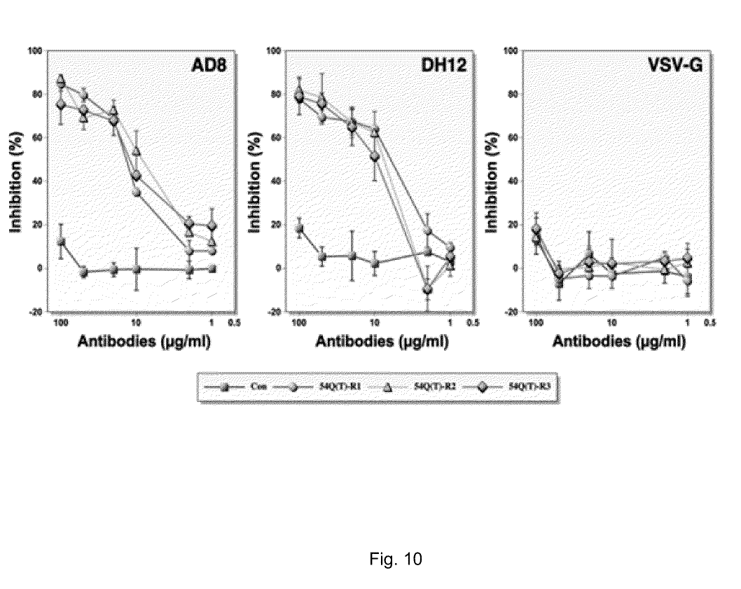

To demonstrate that the virus neutralizing activity was indeed due to antibodies, we purified total IgG from immunized and control rabbit sera, and conducted neutralization assay against two primary HIV-1 isolates. As shown in FIG. 10, IgG from all three immunized rabbits was able to neutralize both HIV-1 pseudoviruses, but not VSV-G pseudovirus. No neutralizing activity was observed for IgG purified from a mock-immunized control rabbit, demonstrating that the neutralizing activity is indeed due to antibodies and that they are specific against HIV-1.

Identification of a Potential Neutralizing Epitope on gp41-54Q

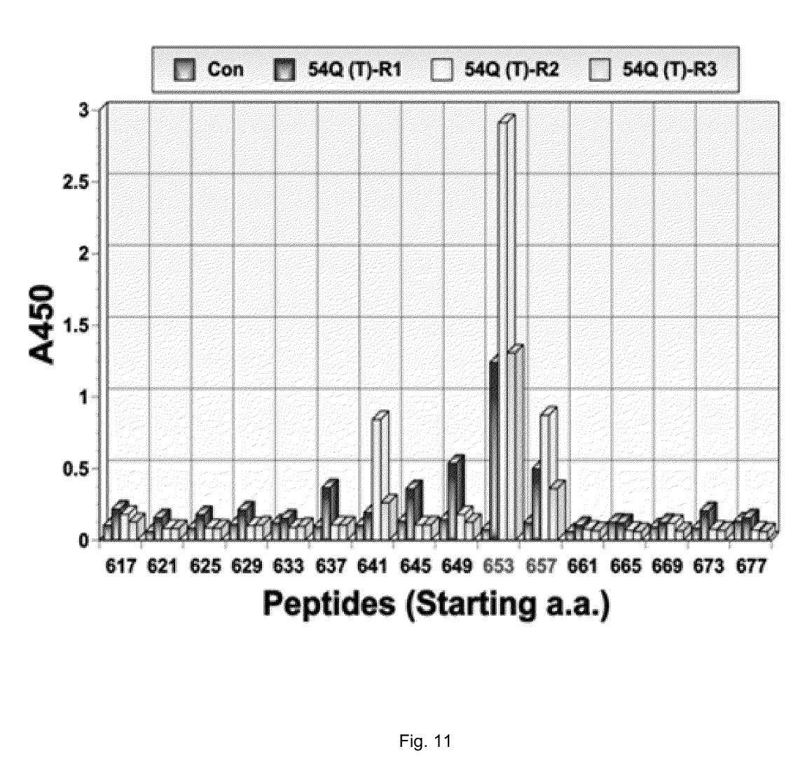

In order to identify possible neutralization epitopes, we performed ELISA with overlapping peptides across the entire length of the antigen. As shown in FIG. 11, there was strong recognition of peptide #653 (QEKNEQELLALDKWA) (SEQ ID NO: 24), especially for rabbit #2, which exhibited the greatest neutralizing activity. This peptide contains the epitope recognize by neutralizing mAb 2F5 (underlined). It should be noted that the immune sera also recognized peptide #657 (EQELLALDKWASLWN) (SEQ ID NO: 25), which is also weakly recognized by 2F5. Thus, these results suggest that gp41-54Q is able to elicit “2F5-like” antibodies.

In addition to linear epitope mapping, we carried out bioinformatic analyses to identify amino acid residues that might be targeted by neutralizing antibodies. The C-terminal 54 amino acid residues of gp41 ectodomain (a.a. 630-683) from viruses subjected to neutralization assays were aligned and a possible correlation between the sequence and neutralization sensitivity was evaluated. Although there are few exceptions, a phylogenetic tree view of aligned sequences revealed fairly clear separation of viruses into neutralization positive and negative (or weakly neutralizing) clusters. Not surprisingly, the sequence of the gp41-54Q immunogen was situated in the middle of the neutralization positive cluster. Four residues showed significant bias between the clusters: positions 659,662,667 and 668. The negative cluster has mostly D/A at 659/662 while the positive cluster is dominated by E/E. For residues 667/668, they are mostly A/S pair for the positive cluster (although there is an E/S subcluster within the positive cluster) while the negative cluster has dominant Asn presence. These results are consistent with those from the overlapping peptide ELISA studies and suggest that our gp41-54Q can elicit neutralizing antibodies that target an epitope that most likely overlaps with 2F5 epitope.

From the above description of the invention, those skilled in the art will perceive improvements, changes and modifications. Such improvements, changes, and modifications are within the skill of the art and are intended to be covered by the appended claims.

Having described the invention, the following is claimed:

1. A vaccine for HIV-1 comprising:
a polypeptide that includes an amino acid sequence that is substantially homologous to about 50 to about 64 consecutive amino acids of a c-terminal portion of the membrane proximal external region of an HIV-1 envelope glycoprotein gp41 ectodomain, the polypeptide being soluble in an aqueous solution and including an immunogenic epitope reactive with at least one anti-HIV antibody; and
a pharmaceutically acceptable carrier.

2. The vaccine of claim 1, the polypeptide having an amino acid sequence that is substantially homologous to SEQ ID NO: 1, SEQ ID NO: 2 or SEQ ID NO: 3.

3. The vaccine of claim 1, further comprising a leader sequence coupled to the N-terminus of the polypeptide.

4. The vaccine of claim 1, the leader sequence having the amino acid sequence of SEQ ID NO: 4, SEQ ID NO: 5, SEQ ID NO: 6, SEQ ID NO: 7, or a combination thereof.

5. The vaccine of claim 1, further comprising a polyhistadine-tag coupled to the C-terminus of the polypeptide.

6. The vaccine of claim 1, the polypeptide having the amino acid sequence of SEQ ID NO: 8, SEQ ID NO: 9, SEQ ID NO: 10, SEQ ID NO: 11, SEQ ID NO: 12, or a combination thereof.

7. The vaccine of claim 1, the aqueous solution having a pH of about 7 to about 8.

8. The vaccine of claim 1, the aqueous solution containing at least one of urea, PBS and HEPES.

9. The vaccine of claim 1, the at least one anti-HIV antibody being selected from the group of antibodies consisting of 2F5, 4E10 and Z13e1.

10. A method for producing a soluble polypeptide comprising a portion of the HIV-1 gp41 ectodomain, the method comprising the steps of:
transfecting a host cell with a vector, the vector including a polynucleotide having a nucleic acid sequence substantially homologous to SEQ ID NO: 13, SEQ ID NO: 14, SEQ ID NO: 15, SEQ ID NO: 16, SEQ ID NO: 17, SEQ ID NO: 18, SEQ ID NO: 19, or SEQ ID NO: 20;
expressing a soluble polypeptide encoded by the nucleic acid sequence; and
purifying the soluble polypeptide, the polypeptide being soluble in an aqueous solution and including an immunogenic epitope reactive with at least one anti-HIV antibody.

11. The method of claim 10 further including the step of solubilizing the polypeptide in the aqueous solution.

12. The method of claim 10, the aqueous solution having a pH of about 7 to about 8.

13. The method of claim 12, the aqueous solution containing at least one of urea, PBS and HEPES.

14. The method of claim 10, the step of purifying the soluble polypeptide further comprising the steps of:
solubilizing the polypeptide in a urea-containing aqueous solution;
renaturing the polypeptide; and
isolating the polypeptide.

15. The method of claim 10, the polypeptide being purified by affinity chromatography, ion exchange chromatography, ultracentrifugation, gel filtration, or a combination thereof.

16. A method for inducing an immune response against HIV or an HIV epitope in a subject, the method comprising the steps of:
administering to the subject a polypeptide in an amount effective to elicit the immune response in the subject;
wherein the polypeptide includes an amino acid sequence that is substantially homologous to about 50 to about 64 consecutive amino acids of a c-terminal portion of the membrane proximal external region of an HIV-1 envelope glycoprotein gp41 ectodomain, the polypeptide being soluble in an aqueous solution and including an immunogenic epitope reactive with at least one anti-HIV antibody;

17. The method of claim 16, the polypeptide having an amino acid sequence that is substantially homologous to SEQ ID NO: 1, SEQ ID NO: 2 or SEQ ID NO: 3.

18. The method of claim 16, further comprising a leader sequence coupled to the N-terminus of the polypeptide.

19. The method of claim 16, the leader sequence having the amino acid sequence of SEQ ID NO: 4, SEQ ID NO: 5, SEQ ID NO: 6, SEQ ID NO: 7, or a combination thereof.

20. The method of claim 16, further comprising a polyhistadine-tag coupled to the C-terminus of the polypeptide.

21. The method of claim 16, the polypeptide having the amino acid sequence of SEQ ID NO: 8, SEQ ID NO: 9, SEQ ID NO: 10, SEQ ID NO: 11, SEQ ID NO: 12, or a combination thereof.

22. The method of claim 16, the aqueous solution having a pH of about 7 to about 8.

23. The method of claim 16, the aqueous solution containing at least one of urea, PBS and HEPES.

24. The method of claim 16, the at least one anti-HIV antibody being selected from the group of antibodies consisting of 2F5, 4E10 and Z13e1.


SEQUENCE LISTING (25 sequences)

SEQ ID NO: 1 - protein, 64 aa, Human immunodeficiency virus
DEIWDNMTWMEWEREISNYTDIIYRLIEESQNQQEKNEQELLALDKWASLWNWFDITNWLWYIK

SEQ ID NO: 2 - protein, 54 aa, Human immunodeficiency virus
EWEREISNYTDIIYRLIEESQNQQEKNEQELLALDKWASLWNWFDITNWLWYIQ

SEQ ID NO: 3 - protein, 54 aa, Human immunodeficiency virus
EWEREIDNYTGLIYTLIEESQNQQEKNEQELLELDKWASLWNWFDITNWLWYIK

SEQ ID NO: 4 - protein, 14 aa, Artificial Sequence
  SYNTHETIC CONSTRUCT
MASMTGGQQMGRGS

SEQ ID NO: 5 - protein, 5 aa, Artificial Sequence
  SYNTHETIC CONSTRUCT
MTSGS

SEQ ID NO: 6 - protein, 5 aa, Artificial Sequence
  SYNTHETIC CONSTRUCT
MVHGS

SEQ ID NO: 7 - protein, 5 aa, Artificial Sequence
  SYNTHETIC CONSTRUCT
MASGS

SEQ ID NO: 8 - protein, 74 aa, Artificial Sequence
  SYNTHETIC CONSTRUCT
MASMTGGQQMGRGSEWEREISNYTDIIYRLIEESQNQQEKNEQELLALDKWASLWNWFDITNWLWYIQHHHHHH

SEQ ID NO: 9 - protein, 74 aa, Artificial Sequence
  SYNTHETIC CONSTRUCT
MASMTGGQQMGRGSEWEREIDNYTGLIYTLIEESQNQQEKNEQELLELDKWASLWNWFDITNWLWYIKHHHHHH

SEQ ID NO: 10 - protein, 65 aa, Artificial Sequence
  SYNTHETIC CONSTRUCT
MTSGSEWEREISNYTDIIYRLIEESQNQQEKNEQELLALDKWASLWNWFDITNWLWYIQHHHHHH

SEQ ID NO: 11 - protein, 65 aa, Artificial Sequence
  SYNTHETIC CONSTRUCT
MVHGSEWEREISNYTDIIYRLIEESQNQQEKNEQELLALDKWASLWNWFDITNWLWYIQHHHHHH

SEQ ID NO: 12 - protein, 65 aa, Artificial Sequence
  SYNTHETIC CONSTRUCT
MASGSEWEREISNYTDIIYRLIEESQNQQEKNEQELLALDKWASLWNWFDITNWLWYIQHHHHHH

SEQ ID NO: 13 - DNA, 192 bp, Human immunodeficiency virus
gacgagatctgggacaacatgacctggatggagtgggagcgcgagatctccaactacaccgacatcatctaccgcctgatcgaggagtcccagaaccagcaggagaagaacgagcaggagctgctggccctggacaagtgggcctccctgtggaactggttcgacatcaccaactggctgtggtacatcaag

SEQ ID NO: 14 - DNA, 162 bp, Human immunodeficiency virus
gagtgggagcgcgagatctccaactacaccgacatcatctaccgcctgatcgaggagtcccagaaccagcaggagaagaacgagcaggagctgctggccctggacaagtgggcctccctgtggaactggttcgacatcaccaactggctgtggtacatccag

SEQ ID NO: 15 - DNA, 162 bp, Human immunodeficiency virus
gagtgggaaagagaaatcgacaactacacaggcttaatatacacattaattgaagaatcgcagaaccagcaagaaaagaatgaacaagaattattagaattagataagtgggcaagtttgtggaattggtttgacataacaaattggctgtggtatataaaa

SEQ ID NO: 16 - DNA, 222 bp, Artificial Sequence
  SYNTHETIC CONSTRUCT
atggctagcatgactggtggacagcaaatgggtcgcggatccgagtgggagcgcgagatctccaactacaccgacatcatctaccgcctgatcgaggagtcccagaaccagcaggagaagaacgagcaggagctgctggccctggacaagtgggcctccctgtggaactggttcgacatcaccaactggctgtggtacatccagcatcaccatcatcaccat

SEQ ID NO: 17 - DNA, 222 bp, Artificial Sequence
  SYNTHETIC CONSTRUCT
atggctagcatgactggtggacagcaaatgggtcgcggatccgagtgggaaagagaaatcgacaactacacaggcttaatatacacattaattgaagaatcgcagaaccagcaagaaaagaatgaacaagaattattagaattagataagtgggcaagtttgtggaattggtttgacataacaaattggctgtggtatataaaacatcaccatcatcaccat

SEQ ID NO: 18 - DNA, 195 bp, Artificial Sequence
  SYNTHETIC CONSTRUCT
atgactagtggatccgagtgggagcgcgagatctccaactacaccgacatcatctaccgcctgatcgaggagtcccagaaccagcaggagaagaacgagcaggagctgctggccctggacaagtgggcctccctgtggaactggttcgacatcaccaactggctgtggtacatccagcatcaccatcatcaccat

SEQ ID NO: 19 - DNA, 195 bp, Artificial Sequence
  SYNTHETIC CONSTRUCT
atggtgcacggatccgagtgggagcgcgagatctccaactacaccgacatcatctaccgcctgatcgaggagtcccagaaccagcaggagaagaacgagcaggagctgctggccctggacaagtgggcctccctgtggaactggttcgacatcaccaactggctgtggtacatccagcatcaccatcatcaccat

SEQ ID NO: 20 - DNA, 195 bp, Artificial Sequence
  SYNTHETIC CONSTRUCT
atggctagcggatccgagtgggagcgcgagatctccaactacaccgacatcatctaccgcctgatcgaggagtcccagaaccagcaggagaagaacgagcaggagctgctggccctggacaagtgggcctccctgtggaactggttcgacatcaccaactggctgtggtacatccagcatcaccatcatcaccat

SEQ ID NO: 21 - DNA, 27 bp, Artificial Sequence
  SYNTHETIC CONSTRUCT
cgcggatccgagtgggagcgcgagatc

SEQ ID NO: 22 - DNA, 48 bp, Artificial Sequence
  SYNTHETIC CONSTRUCT
gatgaattcttaatggtgatgatggtgatgctggatgtaccacagcca

SEQ ID NO: 23 - protein, 12 aa, Artificial Sequence
  SYNTHETIC CONSTRUCT
MASMTGGQQMGR

SEQ ID NO: 24 - protein, 15 aa, Artificial Sequence
  SYNTHETIC CONSTRUCT
QEKNEQELLALDKWA

SEQ ID NO: 25 - protein, 15 aa, Artificial Sequence
  SYNTHETIC CONSTRUCT
EQELLALDKWASLWN